(19) United States
(12) Patent Application Publication
(10) Pub. No.: US 2007/0010845 A1
(43) Pub. Date: Jan. 11, 2007

(54) Directionally controlled expandable device and methods for use

(76) Inventors: Gorman Gong, Santa Clara, CA (US); Avram Allan Edidin, Sunnyvale, CA (US); Reynaldo A. Osorio, Daly City, CA (US); Hugues F. Malandain, Mountain View, CA (US)

Correspondence Address: KILPATRICK STOCKTON LLP - 55461, 1001 WEST FOURTH STREET, WINSTON-SALEM, NC, 27101, US

(21) Appl. No.: 11/177,666

(22) Filed: Jul. 8, 2005

(51) Int. Cl. A61M 29/00

(52) U.S. Cl. 606/192

(57) ABSTRACT

Systems and methods for directionally controlling expansion of an expandable device are described. One such device includes an expandable body comprising a first wall portion and a second wall portion. The first wall portion comprises a high elasticity material. The second wall portion comprises a material having an elasticity lower than the elasticity of the first wall portion. When the body is expanded, expansion of the second wall portion is constrained more than expansion of the first wall portion. Expansion of the body is directed outwardly from the high elasticity first wall portion. Such a device is useful for providing cavities in interior body regions.

### FIELD OF THE INVENTION

The invention relates to systems and methods for directionally controlling expansion of an expandable device useful for providing cavities in interior body regions for diagnostic or therapeutic purposes.

### BACKGROUND OF THE INVENTION

Certain diagnostic or therapeutic procedures require provision of a cavity in an interior body region. For example, as disclosed in U.S. Pat. Nos. 4,969,888 and 5,108,404, a balloon may be deployed to form a cavity in cancellous bone tissue, as part of a therapeutic procedure that fixes fractures or other abnormal bone conditions, both osteoporotic and non-osteoporotic in origin. The balloon or other expandable body may compress the cancellous bone to form an interior cavity. A filling material, such as a bone cement, may be inserted into the cavity in order to provide interior structural support for cortical bone.

This procedure can be used to treat cortical bone, which—due to osteoporosis, avascular necrosis, cancer, trauma, or other disease—is fractured or is prone to compression fracture or collapse. These conditions, if not successfully treated, can result in deformities, chronic complications, and an overall adverse impact upon the quality of life.

As a balloon is expanded during such a procedure, it may not expand in the direction desired by a user of the device. Thus, a demand exists for systems and methods capable of directionally controlling expansion of an expandable device useful for providing cavities in interior body regions.

### SUMMARY OF THE INVENTION

Embodiments of the present invention provide systems and methods for directionally controlling expansion of an expandable device useful for providing cavities in interior body regions. One illustrative embodiment comprises a device having an expandable body comprising a wall having two portions. The first wall portion comprises a high elasticity material. The second wall portion comprises a material having an elasticity lower than the elasticity of the material in the first wall portion. When the expandable body is expanded, expansion of the second wall portion is constrained more than expansion of the first wall portion. As a result, expansion of the expandable body is directed outwardly from the high elasticity first wall portion.

In an illustrative embodiment, the expandable body is coupled to the distal end of an elongate member. A cannula is introduced into an interior body region. The elongate member is inserted through the cannula such that the expandable body is positioned for expanding in a selected direction in the interior body region. The body is then expanded, and the first wall portion expands in the selected direction. As a result, the directed expansion creates a cavity within the interior body region. The cavity can then be filled with a filler material.

Features of a directionally controlled expandable device and methods for use of the present invention may be accomplished singularly, or in combination, in one or more of the embodiments of the present invention. As will be realized by those of skill in the art, many different embodiments of a directionally controlled expandable device and methods for use according to the present invention are possible. Additional uses, advantages, and features of the invention are set forth in the illustrative embodiments discussed in the detailed description herein and will become more apparent to those skilled in the art upon examination of the following.

### BRIEF DESCRIPTION OF THE DRAWINGS

FIG. 1 is a side view of a cannula having an expandable body coupled to the distal end of an elongate member inserted through the cannula in an embodiment of the present invention.

FIG. 2 is an enlarged side view of the expandable body shown in the embodiment in FIG. 1.

FIG. 3 is an elevation (lateral) view of several human vertebrae, with a cannula establishing a path to a vertebral body of one of the vertebrae.

FIG. 4 is a plan (coronal) view of a human vertebra being accessed by a cannula, with portions of the vertebra removed to reveal cancellous bone within a vertebral body.

FIGS. 5A-14A are cross-sectional views of expandable bodies having various configurations of high elasticity wall portions and low elasticity wall portions in embodiments of the present invention.

FIGS. 5B-14B are diagrammatic views of the expanded shapes of the expandable bodies having the cross-sections in the corresponding FIGS. 5A-14A embodiments of the present invention.

FIG. 15A is a cross-sectional view of an expandable body having high elasticity wall portions and low elasticity wall portions and an internal restraint in an embodiment of the present invention.

FIG. 15B is diagrammatic view of the expanded shape of the expandable body having the cross-section in FIG. 15A.

FIG. 16A is a cross-sectional view of an expandable body having high elasticity wall portions and low elasticity wall portions and an internal restraint in an embodiment of the present invention.

FIG. 16B is diagrammatic view of the expanded shape of the expandable body having the cross-section in FIG. 16A.

FIG. 17A is a cross-sectional view of an expandable body having a high elasticity wall portion and a low elasticity wall portion in the configuration of a semi-circle in an embodiment of the present invention.

FIG. 17B is diagrammatic view of the expanded shape of the expandable body having the cross-section in FIG. 17A.

FIG. 18A is a cross-sectional view of an expandable body having a low elasticity wall portion along a portion of the length of one side of the body in an embodiment of the present invention.

FIG. 18B is diagrammatic view of the expanded “bean” shape of the expandable body having the cross-section in FIG. 18A.

FIG. 19 is a plan view of the expandable body having the cross-section shown in FIG. 6A in expanded shape in a vertebral body, with portions of the vertebral body removed to reveal compression of cancellous bone in a selected direction.

FIG. 20 is a side view of the expandable body in expanded shape in a vertebral body shown in FIG. 19.

FIG. 21 is a plan view of the expandable body having the cross-section shown in FIG. 7A in expanded shape in a vertebral body, with portions of the vertebral body removed to reveal compression of cancellous bone in a selected direction.

FIG. 22 is a side view of the expandable body in expanded shape in a vertebral body shown in FIG. 21.

FIG. 23 is a plan view of a human vertebra being accessed by cannulae bilaterally, with portions of the vertebra removed to reveal cancellous bone within a vertebral body.

FIG. 24 is a plan view of the expandable body having the cross-section shown in FIG. 18A in expanded shape in a vertebral body, with portions of the vertebral body removed to reveal compression of cancellous bone in a selected direction from a unilateral approach.

FIG. 25 is a flow chart of a method according to an embodiment of the present invention.

### DETAILED DESCRIPTION

Embodiments of the present invention provide systems and methods for directionally controlling expansion of an expandable device useful for providing cavities in interior body regions. The systems and methods embodying the invention can be adapted for use in many suitable interior body regions, wherever the formation of a cavity within or adjacent one or more layers of tissue may be required for a therapeutic or diagnostic purpose. The illustrative embodiments show the invention in association with systems and methods used to treat bones. In other embodiments, the present invention may be used in other interior body regions or types of tissues.

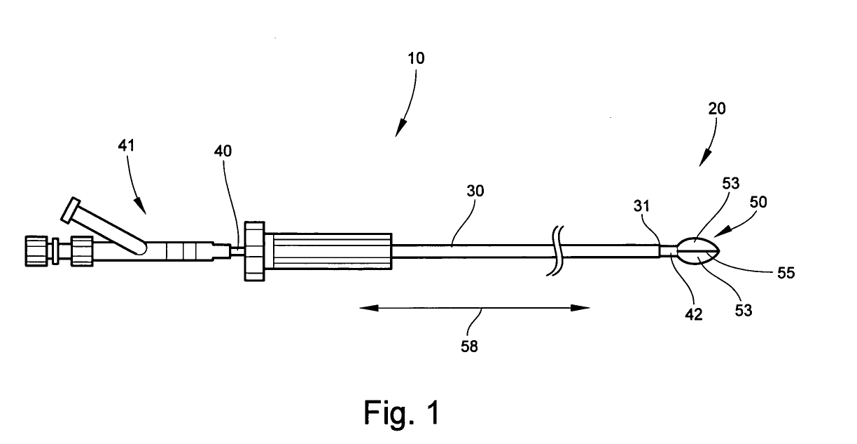

Referring now to the figures, FIG. 1 is a view of a system 10 according to an embodiment of the present invention configured to allow an user to provide a cavity in a targeted treatment area in an interior body region. The system 10 includes a directionally controlled expandable device 20 configured to be used in a kyphoplasty procedure. Kyphoplasty is a minimally invasive surgical procedure for restoring height to an injured or diseased vertebra. In a kyphoplasty procedure, after a cavity is formed in a vertebral body, a filler material is introduced into the resulting cavity to provide increased height and stability to the vertebra.

The system 10 comprises a cannula 30 comprising a proximal end and a distal end 31. The cannula 30 may be fabricated from a material selected to facilitate advancement and rotation of an elongate member 40 movably disposed within the cannula 30. The cannula 30 can be constructed, for example, using standard flexible, medical grade plastic materials, such as vinyl, polyamides, polyolefins, ionomers, polyurethane, polyether ether ketone (PEEK), polycarbonates, polyimides, and polyethylene tetraphthalate (PET). The cannula 30 can be constructed as a bi-layer or a tri-layer of one or more of these materials. The cannula 30 can also comprise more rigid materials to impart greater stiffness and thereby aid in its manipulation and torque transmission capabilities. More rigid materials useful for this purpose include stainless steel, nickel-titanium alloys (such as Nitinol), and other metal alloys.

The system shown in FIG. 1 comprises the elongate member 40 movably disposed within the cannula 30. The elongate member 40 may be made from a resilient inert material providing torsion transmission capabilities, for example, stainless steel, a nickel-titanium alloy such as Nitinol, and other suitable metal alloys. In other embodiments, the elongate member 40 may be fashioned from a variety of suitable materials, such as a carbon fiber, a glass, or a flexible material, for example, as a plastic or rubber. In an embodiment comprising a flexible elongate member 40, the elongate member 40 may be formed, for example, from twisted wire filaments, such stainless steel, nickel-titanium alloys (such as Nitinol), and other suitable metal alloys.

The elongate member 40 shown is hollow, allowing for movement of a flowable material, for example, a liquid or a gas, through the elongate member 40. The elongate member 40 may comprise a handle (not shown) at its proximal end 41 to aid in gripping and maneuvering the elongate member 40. For example, in an embodiment, such a handle can be formed from a foam material and secured about the proximal end 41 of the elongate member 40.

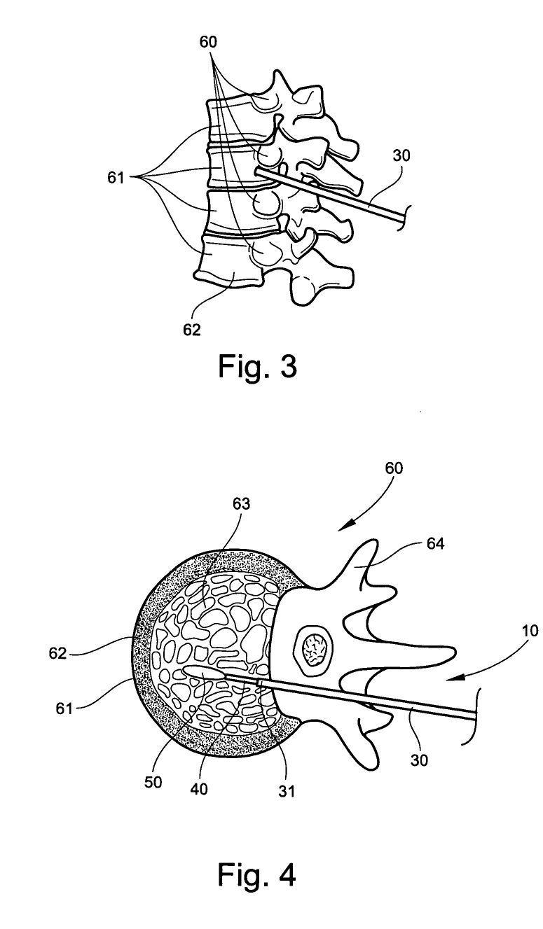

The system shown in FIG. 1 comprises a directionally controlled expandable device 20 configured to be deployed adjacent a tissue in the targeted treatment area via the cannula 30. An expandable body 50 is disposed at the distal end 42 of the elongate member 40, and is thus configured to slide and rotate within the cannula 30. In an embodiment, the expandable body 50 may be configured to be deployed within a treatment area through a percutaneous path established by the cannula 30. For example, the expandable body 50 may be deployed within cancellous bone tissue 63 in a vertebral body 61, as shown in FIGS. 3-4.

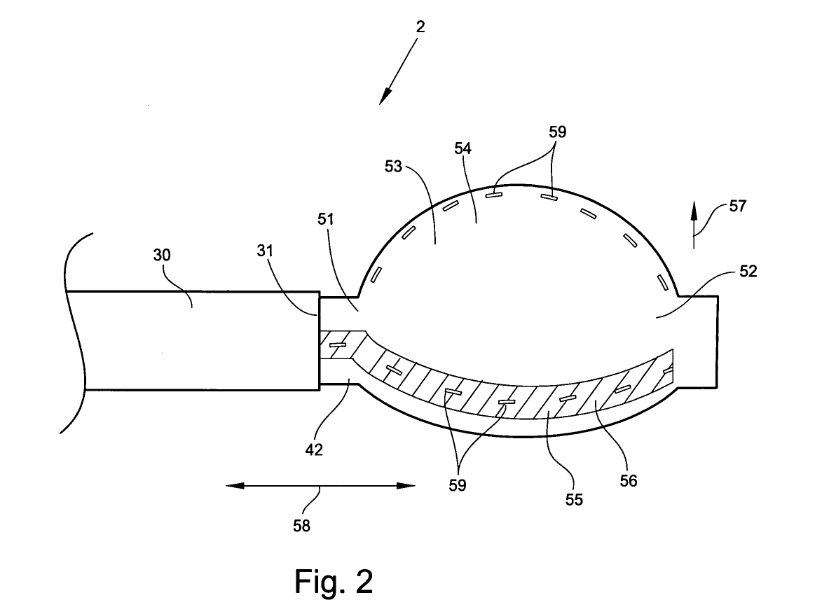

The expandable body 50 may be expanded by movement of a flowable material through the hollow elongate member 40 and into the interior of the expandable body 50. In the embodiment shown in FIGS. 1-2, once the expandable body 50 has been inserted through the cannula 30 to a point beyond the distal end 31 of the cannula 30, a flowable material is introduced through the elongate member 40 to expand the expandable body 50. The expandable body 50 may be contracted by withdrawing the flowable material out of the expandable body 50 through the bore of the elongate member 40. The elongate member 40 and the contracted expandable body 50 may then be withdrawn through the cannula 30.

The expandable body 50 is configured to constrain expansion in selected portions of the expandable body 50 as it expands. The expandable body 50 may comprise an inflatable balloon tube 51, as shown in FIG. 1-2. The expandable body 50 comprises a wall 52 having a first wall portion 53 comprising a high elasticity material 54 and a second wall portion 55 comprising a material 56 having an elasticity lower than the elasticity of the first wall portion 53. Elasticity is defined as the condition or property of returning to an initial form or state following deformation. Deformation is defined as a change in shape due to an applied force, such as the force exerted on a balloon material when the balloon is expanded. Elasticity refers to the degree to which a material is capable of deforming and returning to an initial form or state following deformation. A high elasticity material will deform and return to an initial form or state following deformation more readily than will a low elasticity material. As a result, expansion of the second wall portion 55 is constrained more than expansion of the first wall portion 53 such that expansion of the body 50 is directed outwardly 57 from the higher elasticity first wall portion 53. The first wall portion 53 and the second wall portion 55 extend along an elongated axis 58 of the expandable body 50. Since expansion of the lower elasticity second wall portion 55 is constrained more than expansion of the higher elasticity first wall portion 53, expansion of the body 50 is constrained lengthwise along the elongated axis 58. Accordingly, the direction and degree of expansion of the expandable body 50 can be controlled.

In an embodiment of such an expandable body 50, the high elasticity material 54 may comprise a low durometer (softer) material, and the low elasticity material 56 may comprise a high durometer (harder) material. Durometer is defined as a measure of material hardness or the relative resistance to indentation of various grades of polymers. A higher durometer material may be more resistant to elastic deformation than a lower durometer material. Accordingly, expansion of a high durometer wall material may be constrained more than expansion of a low durometer wall material such that expansion of the expandable body 50 is directed outwardly from the lower durometer wall portion. As a result, a differential in durometer of materials in selected wall portions can be used to control the direction and degree of expansion of the expandable body 50.

In one embodiment of the present invention, at least a portion of the elongate member 40 may comprise one or more radiographic markers (not shown). As shown in the embodiment in FIG. 2, the expandable body 50 may comprise one or more radiographic markers 59 to allow radiographic visualization of the expandable body 50 in an interior body region. In alternative embodiments, the first and/or second wall portions 53, 55, respectively, of the expandable body 50 may be formed from a radiopaque material (discussed below).

The elongate member 40, and thereby the expandable body 50, may be in communication with a controller (not shown), such as a slide controller, a pistol grip controller, a ratcheting controller, a threaded controller, or any other suitable type of controller that can be configured to permit a user of the device to control the extent to which the expandable body 50 extends beyond the distal end 31 of the cannula 30. Such a controller may permit a user of the device 20 to provide rotational torque and thereby control rotation of the elongate member 40 and the expandable body 50.

In the embodiment shown in FIGS. 1-2, the system 10, and in particular the expandable body 50, may be used to provide a cavity in an interior body region. A user of the system causes the expandable body 50 to expand and provide force to surrounding tissues to create a cavity of a desired shape and dimension. In embodiments, the expandable body 50 comprises one or more wall portions 53, 55 having an elasticity relatively lower or higher than one or more other wall portions 53, 55, as described herein. As such, expansion of the expandable body 50 can be directed to create a cavity having a preferred size and shape, while avoiding pressure to undesired areas.

Once a cavity is created in the target treatment area, the expandable body 50 may be contracted and removed from the interior body region through the cannula 30. After the expandable body 50 is removed, a material or filler, such as a bone cement, may then be used to fill the cavity provided by the system 10. Use of a filler material may be beneficial in certain treatment areas, for example, in a vertebra where the system 10 is used to restore height to a vertebral body (see FIGS. 20 and 22, discussed below).

Referring now to FIGS. 3-4, an elevation (lateral) view of several human vertebrae 60 is shown, with a cannula 30 establishing a percutaneous path along its elongated axis 58 to a vertebral body 61 of one of the several vertebrae 60. The vertebral body 61 extends on the anterior (i.e., front or chest) side of the vertebrae 60. The vertebral body 61 comprises an exterior formed from compact cortical bone 62. Cortical bone (62) is defined as bone consisting of, or relating to, cortex, or outer layer of a bony structure. The cortical bone 62 encloses an interior volume of reticulated cancellous 63, or spongy, bone (also called medullary bone or trabecular bone). Cancellous bone (63) is defined as bone having a porous structure having many small cavities or cells in it.

Due to various traumatic or pathologic conditions, such as osteoporosis, a vertebral body 61 can experience a vertebral compression fracture (VCF). In such conditions, cancellous bone 63 can be compacted, causing a decrease in height of the vertebra 60. In a VCF in particular, vertebral height is lost in the anterior region of the vertebral body 61. The user of the system 10 may utilize it to provide a cavity within the vertebral body 61, and to restore height to the vertebral body 61 lost when a fracture occurred.

Systems and methods according to the present invention are not limited in application to human vertebrae 60, and may be used to provide cavities within other parts of a living or non-living organism. For example, in embodiments, the system 10 can be deployed in other bone types and within or adjacent other tissue types, such as in a vertebral disc, an arm bone, a leg bone, a knee joint, etc.

The vertebral body 61 is in the shape of an oval disc. As FIGS. 3-4 show, access to the interior volume of the vertebral body 61 can be achieved, for example, by drilling an access portal through a rear side of the vertebral body 61 (a postero-lateral approach). The portal for the postero-lateral approach enters at a posterior side of the vertebral body 61 and extends anteriorly into the vertebral body 61. Alternatively, access into the interior volume of a vertebral body 61 can be accomplished by drilling an access portal through one or both pedicles 64 of the vertebra 60. This is known as a transpedicular approach.

FIG. 4 shows a vertebra 60 being accessed by the system 10 according to an embodiment of the present invention. The vertebra 60 is shown with portions removed to reveal cancellous bone 63 within the vertebral body 61. The user of the system 10 may slide the elongate member 40 and expandable body 50 axially, or lengthwise along the elongated axis 58, within the cannula 30 to deploy the expandable body 50 in the targeted treatment site. When deployed at the site, the user can extend the expandable body 50 outside the distal end 31 of the cannula 30 adjacent cancellous bone tissue 63 within the vertebral body 61. The user may rotate the elongate member 40, and thereby the expandable body 50, to position the expandable body 50 for directed expansion in the targeted treatment area. Once moved beyond the distal end 31 of the cannula 30, the expandable body 50 may be expanded from a contracted state to an expanded state to provide a cavity within the cancellous bone 63.

Systems and methods of the present invention comprise an expandable body 50, such as the inflatable balloon tube 51 shown in FIG. 2, that are adapted to assume an expanded geometry having a desired configuration when used. Such an expandable body 50 can provide a cavity 81 inside the vertebral body 61 whose configuration is optimal for supporting the bone.

Conventional inflatable balloons become essentially spherical when inflated, creating a generally spherical cavity. Filling a spherical cavity with filler material results in single points of contact on vertebral body 61 surfaces (similar to a circle inside a square, or a sphere inside a cylinder). As a result, such spherical shapes do not typically permit a filler material to support the spine adequately. The directionally-controlled expansion of an expandable body 50 of the present invention creates a preferred shape in a cavity which, when filled with filler material, desirably distributes the load transferred from the vertebral body 61 surfaces to the hardened filler material, ultimately strengthening the spine. Moreover, irregularly-shaped cavities 81 formed by embodiments of the present invention provide shapes, which when filled by filler material can reduce the opportunity for the filler material to shift or displace within the vertebral body 61 under compressive loading of the spine and thereby provide enhanced stability.

Another advantage of an embodiment of the present invention is that embodiments of an expandable body 50 can optimally expand to a desired shape rather than simply towards areas of lowest bone density. That is, expansion of the body 50 can be controlled even when encountering areas in the bone of varying resistance.

Certain injuries and/or diseases cause anatomical malformations along only portions of a spherical shape. For example, vertebral compression fractures often result in collapse of the affected vertebra 60 in a more or less vertical orientation. In reducing such a vertebral compression fracture, it may be desirable to compress cancellous bone 63 only in the direction of collapse. If a vertebral compression fracture is oriented in a vertical direction, expansion of an expandable body 50 according to the present invention can be limited to the vertical direction only. Such a directionally controlled expandable device 20 would allow most of the force of expansion to be directed toward the endplates between affected vertebral bodies 61, thereby increasing the mechanical capability of the expandable body 50 to reduce the fracture. Thus, another advantage of the present invention is that embodiments of an expandable body 50 can move the top and bottom of the vertebral bodies 61 (i.e., the upper and lower vertebral end plates) toward a more normal anatomical position to restore height.

Another advantage is that certain embodiments of the present invention can achieve directed expansion of an expandable body 50 into desired areas while avoiding expansion into areas that are not affected by injury or disease. For example, in a vertebral body 61, the expansion can be prevented from entering an area not affected by a compression fracture. As a result, the outer dimensions of the sides of the vertebral body 61 can be maintained by avoiding fracturing the cortical sidewalls of the vertebral body 61 or by moving already fractured bone in the sidewalls.

Embodiments of an expandable body 50 according to the present invention include wall portions 53, 55 having elasticities 54, 56 sufficiently different to allow the body 50 to differentially expand when under internal pressure. In use, such expandable bodies 50 are able to expand preferentially along one or more axes so as to deliver a greater force and/or displacement of cancellous bone 63 toward one direction versus another.

In one such embodiment, the expandable body 50 comprises a wall 52 having a first wall portion 53 comprising a high elasticity material 54 and a second wall portion 55 comprising a material 56 having an elasticity lower than the first wall portion 53 elasticity. In an illustrative embodiment, the high elasticity material 54 in the first wall portion 53 can comprise a low durometer material, and the lower elasticity material 56 in the second wall portion 55 can comprise a high durometer material. Reference to the durometer, or hardness, of one material is made relative to the durometer, or hardness, of another material. For example, in embodiments of an expandable body 50, a high durometer material wall portion has a higher durometer, or is harder and less pliable, relative to another wall portion comprising a lower durometer, or softer, material.

Polymers such as polyurethanes are available in different hardnesses, according to a hardness, or durometer, scale used in plastics. For example, a durometer of 90A is a degree of hardness on the “A” durometer scale. A material having 90B durometer rating would be harder than a material having a 90A durometer rating. The lower the durometer scale rating, the softer and more pliable the material. For example, the lower the durometer scale rating of a material used in wall portions 55 having higher durometer rated materials 56, the more the expandable body 50 would elongate along an axis 58 in the longitudinal direction. In addition, the amount of increase in expansion force on the softer portions 53 of the wall 52 relate to the durometer of the harder portions 55 of the wall 52. The higher the durometer of the harder portions 55, the greater the increase in expansion force on the softer portions 53.

The expandable body wall 52 can have one or more wall portions 55, or “stripes,” of less elastic material 56 disposed in the longitudinal direction along the elongated axis 58 of the device 20. When expanded, the portions 55 of the expandable body wall 52 comprising lower elasticity material 56 do not stretch as much as the portions 53 of the expandable body wall 52 comprising higher elasticity material 54. Thus, the “stripes,” or longitudinal portions 55 of less elastic material 56, in the expandable body wall 52 are constrained during expansion relative to the wall portions 53 of more elastic material 54. As a result, the direction of expansion about the circumference of the expandable body 50 can be controlled. Embodiments of the expandable body wall portions 55 made with low elasticity material 56 provide the advantage of greater torque control from the attached elongate member 40, or catheter, allowing easier radial, or rotational, movement of the expandable body 50.

The amount of directionality provided by wall portions 55 of lower elasticity material 56 can be adjusted by making those wall portions 55 either more broad or more narrow. A broader wall portion 55 of low elasticity material 56 would force the expandable body 50 to expand less in the direction toward which that wall portion 55 is oriented than a more narrow wall portion 55 of material 56 having the same elasticity. Location of placement of low elasticity wall portions 55 at selected locations around the circumference of the expandable body 50 can provide additional directional control of expansion. For example, two wall portions 55 of low elasticity material 56 located on the same half of a tube circumference would allow expansion from that half of the tube only in the direction outward 57 from the higher elasticity material portion 53 between the two low elasticity material portions 55. In embodiments, multiple wall portion stripes 55 of low elasticity material 56 can be located about the circumference of the expandable body 50. In this way, expansion of the body 50 can be directed from multiple higher elasticity material wall portions 53 toward multiple and more discrete target areas. Directional control of expansion allows the expandable body 50 to expand into non-spherical shapes.

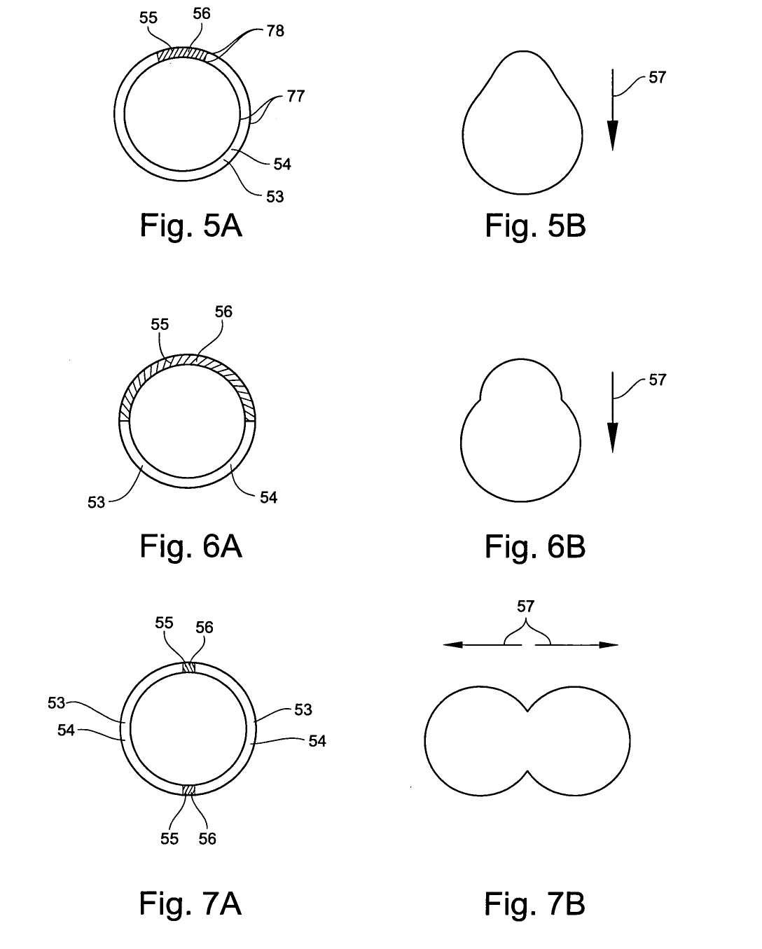

As shown in FIGS. 5-18, embodiments of a directionally-controlled expandable body of the present invention can comprise various cross-sections, for example, round, non-round and profiled cross-sections. For example, FIG. 5A shows a first wall portion 53 (high elasticity material 54) comprising more that three fourths of the cross-section of an expandable body, and a second wall portion 55 (low elasticity material 56) comprising less than one fourth and located on one side of the cross-section. FIG. 5B shows the shape and direction 57 of expansion of the embodiment in FIG. 5A outward from the first wall portion 55. This configuration provides an ovoid-shaped expansion.

FIG. 6A shows a first wall portion 53 (high elasticity material 54) and a second wall portion 55 (low elasticity material 56) each comprising approximately half of the cross-section of an expandable body. FIG. 6B shows the shape and direction 57 of expansion of the embodiment in FIG. 6A outward from the first wall portion 55. This configuration provides a substantially rounded expansion beginning from the edges of the second wall portion 55. As such, the embodiment of an expandable body in FIG. 6A provides a differently shaped (and directed) expansion than the embodiment in FIG. 5A.

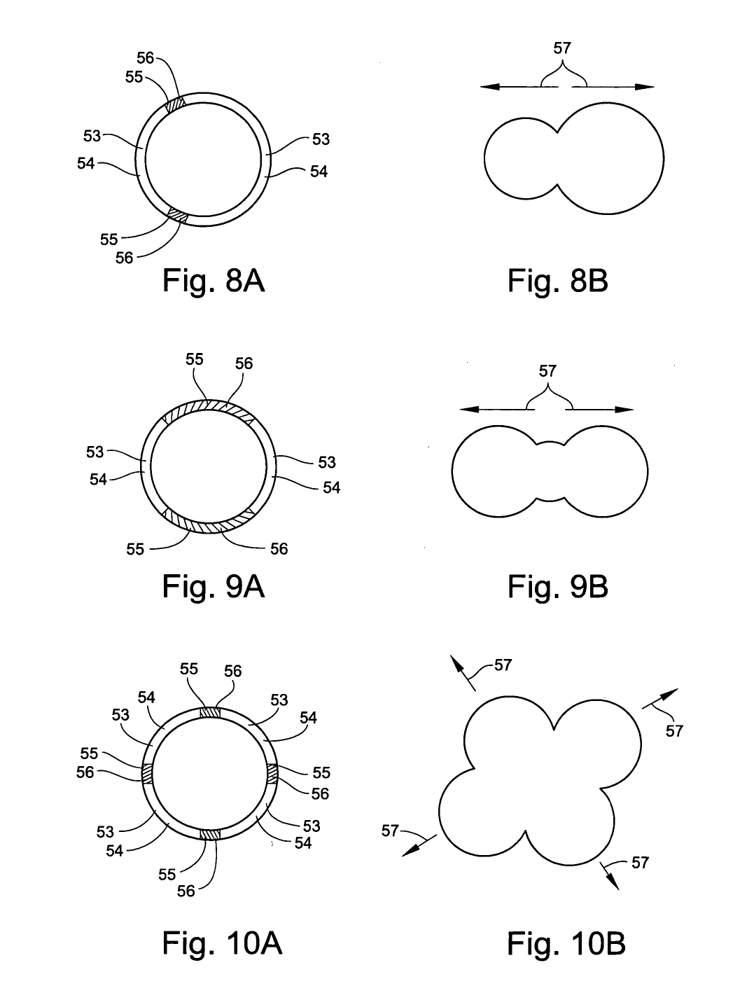

FIG. 7A shows two first wall portions 53 (high elasticity material 54) comprising the large majority of the cross-section of an expandable body, and two second wall portions 55 (low elasticity material 56) each comprising a relatively small portion on opposite sides of the cross-section at the “6” and “12” clock positions (if a clock face was overlaid onto the cross-section). FIG. 7B shows the shape and direction 57 of expansion of the embodiment in FIG. 7A outward from constrained points of the second wall portions 55. This configuration provides an expansion having a “figure 8” shape.

FIG. 8A shows two first wall portions 53 (high elasticity material 54) comprising the large majority of the cross-section of an expandable body, and two second wall portions 55 (low elasticity material 56) each comprising a relatively small portion at the “7” and “11” o'clock positions of the cross-section. FIG. 8B shows the shape and direction 57 of expansion of the embodiment in FIG. 8A outward from constrained points of the second wall portions 55. This configuration provides an expansion having an uneven “figure 8” shape.

FIG. 9A shows two first wall portions 53 (high elasticity material 54) comprising the majority of the cross-section of an expandable body, and two second wall portions 55 (low elasticity material 56) comprising the portions of the cross-section between the “5” and “7” o'clock positions and between the “11” and “1” o'clock positions of the cross-section. FIG. 9B shows the shape and direction 57 of expansion of the embodiment in FIG. 9A outward from constrained second wall portions 55. This configuration provides an expansion having a “shortened dumbbell” shape.

FIG. 10A shows four first wall portions 53 (high elasticity material 54) comprising the majority of the cross-section of an expandable body, and four second wall portions 55 (low elasticity material 56) comprising the portions of the cross-section at the “3,” “6,” “9,” and “12” o'clock positions of the cross-section. FIG. 10B shows the shape and direction 57 of expansion of the embodiment in FIG. 10A outward from constrained second wall portions 55. This configuration provides an expansion having a “cloverleaf” shape.

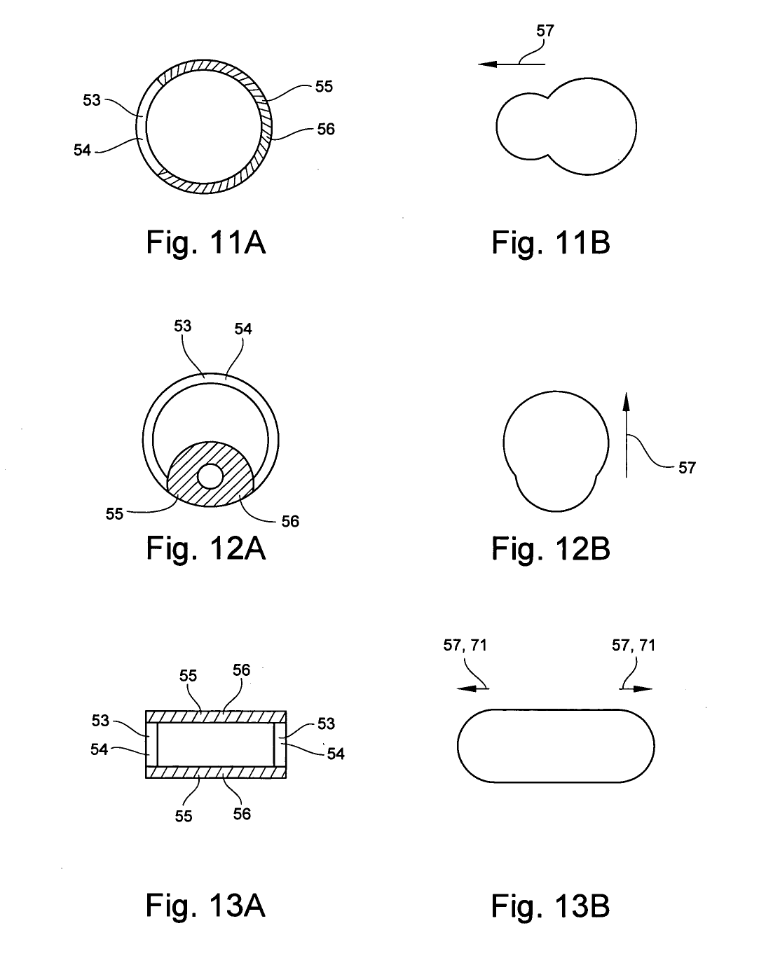

FIG. 11A shows a first wall portion 53 (high elasticity material 54) comprising approximately one fourth of the cross-section of an expandable body and a second wall portion 55 (low elasticity material 56) comprising approximately three fourths of the cross-section. FIG. 11B shows the shape and direction 57 of expansion of the embodiment in FIG. 11A outward from the first wall portion 55. This configuration provides an expansion having a shape largely constrained by the second wall portion 55 and a small, rounded shape expanded from the area of the first wall portion 53.

FIG. 12A shows a first wall portion 53 (high elasticity material 54) comprising more that three fourths of the cross-section of an expandable body, and a second wall portion 55 (low elasticity material 56) comprising less than one fourth and located on one side of the cross-section. The second wall portion 55 extends inwardly into the bore of the expandable body in a semi-circular shape. FIG. 12B shows the shape and direction 57 of expansion of the embodiment in FIG. 12A outward from the first wall portion 55. This configuration provides an expansion having a shape similar to that of a light bulb.

FIG. 13A shows two first wall portions 53 (high elasticity material 54) each comprising opposite sides of a rectangular-shaped expandable body cross-section, and two second wall portions 55 (low elasticity material 56) each comprising opposite sides of the rectangular-shaped cross-section that are shorter than the two first wall portion sides. FIG. 13B shows the shape and direction 57 of expansion of the embodiment in FIG. 13A outward from the first wall portions 55. This configuration provides an oblong-shaped expansion.

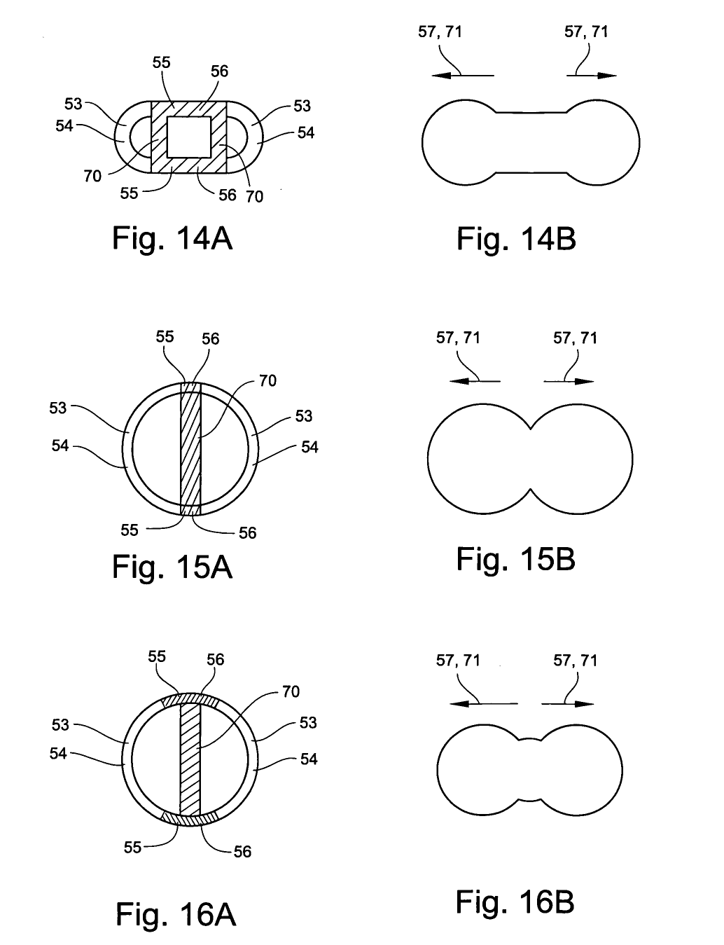

Embodiments of an expandable body according to the present invention can achieve directionally-controlled expansion without using additional structures in the interior of the body. However, in embodiments, the expandable body 50 comprising wall portions 53, 55 comprising differential elasticities can be configured to include an internal restraint. For example, FIGS. 14A-16A shown cross-sections of an expandable body having an internal restraint 70.

FIG. 14A shows two first wall portions 53 (high elasticity material 54) each comprising opposite sides of an expandable body having a partially flattened cross-section, and a second wall portion 55 (low elasticity material 56) in the form of a square, two sides of which are contiguous with the wall of the expandable body and two sides of which form internal restraints 70 connecting opposite sides of the body wall. FIG. 14B shows the shape and direction 57 of expansion of the embodiment in FIG. 14A outward from the first wall portions 55 and in the opposite directions 71 of expansion away from internal restraint 70. This configuration provides an expansion having an “elongated dumbbell” shape.

FIG. 15A shows two first wall portions 53 (high elasticity material 54) each comprising opposite sides of an expandable body cross-section, and two second wall portions 55 (low elasticity material 56) comprising the portions of the cross-section around the “6” and “12” o'clock positions of the cross-section. The internal restraint 70 connects the sides of the body wall adjacent the two second wall portions 55. FIG. 15B shows the shape and direction 57 of expansion of the embodiment in FIG. 15A outward from the first wall portions 55 and in the opposite directions 71 of expansion away from internal restraint 70. This configuration provides an expansion having an “figure 8” shape.

Directionally-controlled expansion of an expandable body can be accomplished with a dual web internal restraint in which expansion control is bi-directional. For example, the Elevate™ inflatable balloon tamp (IBT), which includes a dual web balloon, is disclosed in U.S. Patent Publication No. 2003/0032963. This publication discloses such a dual-web IBT as comprising an uninflated cross-section having a round outer wall and two adjacent inner walls connecting the outer wall across the diameter of the circular shape. This configuration provides three hollow chambers inside the balloon. The two outer chambers have semi-circular shapes and are inflatable. When inflated, each semi-circular chamber moves in opposite directions. The inner walls, or webs, serve as internal expansion restraints during inflation. The internal walls undergo only limited elastic and/or plastic deformation during inflation, thereby maintaining the approximate original balloon diameter at the points where the inner walls are connected to the outer wall. However, the balloon outer wall is not as significantly restrained from expanding in the directions transverse to the internal walls. Thus, the balloon can expand substantially more in one direction than in a transverse direction, for example, more in the vertical direction than in the horizontal direction, resulting in a cross-sectional shape that is generally ovoid or somewhat similar to a “figure 8.”

Such a dual web internal restraint can control expansion in a bi-directional manner. Embodiments of an expandable body of the present invention provide further directional control of expansion not limited to two (opposite) directions. For example, as shown in FIG. 16A, two first wall portions 53 (high elasticity material 54) each comprise opposite sides of an expandable body cross-section, and two second wall portions 55 (low elasticity material 56) comprise the portions of the cross-section around the “6” and “12” o'clock positions of the cross-section. The internal restraint 70 connects the sides of the body wall adjacent the two second wall portions 55. FIG. 16B shows the shape and direction 57 of expansion of the embodiment in FIG. 16A outward from the first wall portions 55 and in the opposite directions 71 of expansion away from internal restraint 70. This configuration provides an expansion having an “elongate figure 8” shape.

Internal restraints 70 can include, for example, mesh work, webbing, membranes, partitions or baffles, a winding, spooling or other material laminated to portions of the balloon body, and continuous or non-continuous strings across the interior of the expandable body 50 held in place at specific locations. In addition, as shown in FIG. 2, the low elasticity wall portions 55 of the expandable body 50 of the present invention provide improved control of lengthwise expansion along the elongated axis 58 of the expandable body 50.

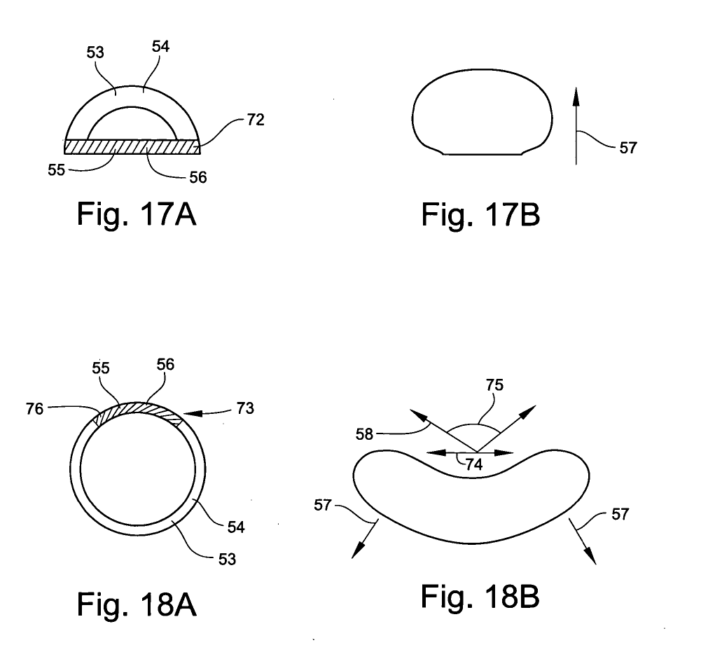

Embodiments of an expandable body of the present invention can be configured to function in a manner similar to expandable bodies having an external restraint. For example, FIG. 17A shows a first wall portion 53 (high elasticity material 54) comprising a semi-circular cross-section of an expandable body, and a second wall portion 55 (low elasticity material 56) comprising the length of the diameter of the semi-circular cross-section. In use, the second wall portion 55 acts as a substantially rigid surface 72. FIG. 17B shows the shape and direction 57 of expansion of the embodiment in FIG. 17A outward from the first wall portion 55. This configuration provides an expansion having an ovoid shape, the expansion occurring primarily in one direction away from the axis of the second wall portion 55. The second wall portion 55 can also prevent compression by the expanding body of anatomical structures behind the second wall portion 55 (substantially rigid surface 72).

In another embodiment of an expandable body of the present invention, FIG. 18A shows a first wall portion 53 (high elasticity material 54) comprising more that three fourths of the cross-section of the expandable body, and a second wall portion 55 (low elasticity material 56) comprising less than one fourth and located on one side of the cross-section. In this embodiment, the second wall portion 55 is a non-compliant material 76 located on one side 73 of the wall 52 and extends the length 74 along the elongated axis 58 of the expandable body 50, which is less than the entire length of the expandable body 50. In this way, when expanded as shown in FIG. 18B, the body 50 expands in an asymmetric, “bean-shaped” or “banana-shaped” fashion, thereby providing expansion of the body 50 outwardly 57 and opposite from the center of the length 74 of the second wall portion 55. The embodiment of the expandable body 50 whose cross-section is shown in FIG. 18A expands at an angle 75 from the elongated axis 58. The angle the expandable body 50 curves from the elongated axis 58 is in the range of 30-90 degrees.

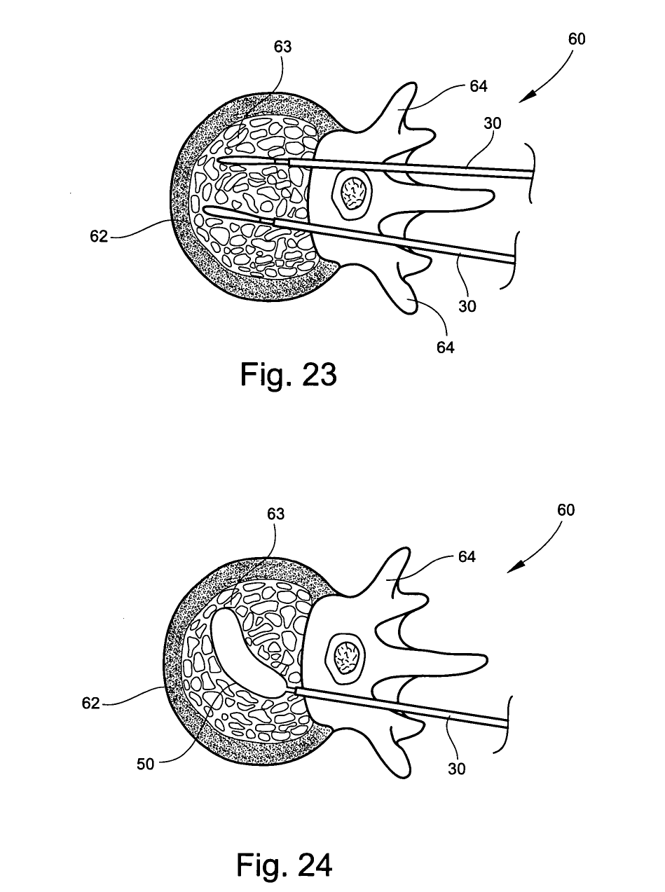

FIG. 23 is a plan view of a human vertebra 60 being accessed bilaterally across pedicles 64 by cannulae 30, with portions of the vertebra 60 removed to reveal cancellous bone 63 within the vertebral body 62. The expandable body 50 is generally deployed via the elongate member 40 across the pedicle 64 on both sides of the vertebra 60. When accessing the vertebral body 61 via the pedicle 64, the expandable body 50 is positioned lateral to the midline of the vertebra 60, or the disc when used for endplate extraction. In both cases, a bilateral approach is necessary.

As shown in FIG. 24, the embodiment in FIGS. 18A and 18B of the expandable body 50 having the cross-section shown and extending the length 74 is inserted in a typical manner using a trans-pedicular approach. When expanded, the expandable body 50 expands to a “bean” shape and curves at the angle 75 (shown in FIG. 18B) such that the body 50 expands beyond one side of the vertebral body 61. The expandable body curves from the elongated axis 58 at an angle in the range of 30-90 degrees. As a result, although the expandable body 50 is inserted along the elongated axis 58 in line with the expandable member 40 when not expanded, the body can be directionally expanded in a curve to compress the cancellous bone 63 on the side of the vertebral body 61 contralateral to the insertion point. Such a “bean-shaped” expandable body 50 would allow a physician to access the vertebral body 61 with a unilateral approach and reach areas not directly aligned with the access trajectory. Such a method would provide access to portions of the vertebral body 61 not reachable when an expandable body cannot be inserted in a direct line across the midline of the vertebral body 61. Used in a unilateral procedure, the expandable body 50 having such a “bean-shaped” expansion would allow a less invasive procedure than a conventional bilateral approach, and would decrease cost by eliminating the need for a second expandable device.

In another embodiment of the present invention, an expandable body 50 comprises one or more wall portions 53 comprising a high elasticity material 54 and having a thickness 77 (as shown in FIG. 5A). The expandable body 50 comprises one or more wall portions 55 comprising a relatively lower elasticity material 56 and having a thickness 78 (as shown in FIG. 5A). In this embodiment, thickness 78 of the low elasticity wall portion(s) 55 is different than the thickness 77 of the higher elasticity wall portion(s) 53. The greater the thickness, or depth, of the low elasticity material wall portion 55, the greater amount of low elasticity material 56 in the wall portion 55. Thus, the thicker a low elasticity material wall portion 55, the greater the rigidity of that wall portion 55. As a result, portion(s) of the wall 52 of the expandable body 50 having an increased thickness stretch less than less thick portion(s) of the wall 52. Accordingly, thickness variation in embodiments of the expandable body 50 can provide additional means for directionally controlling expansion of the body 50.

The amount of low elasticity material 56 in wall portion(s) 55 should be controlled so as to not diminish the elasticity characteristics of the high elasticity material wall portions 53. That is, the total amount of low elasticity material 56 used to achieve a degree of inelasticity should be balanced with elasticity characteristics of the expandable body 50 in the high elasticity portions so that the body 50 can be expanded to a desired shape and dimension.

Expandable bodies 50 of the present invention can comprise low elasticity wall portions 55 made from, for example, polyurethanes, polyolefins (polyethylenes, polypropylenes, etc.), polyamides, acrylics, polyvinyl compounds, polyesters, polyethers, polycarbonates, polyether therephthalate, polyketones, and any of these materials combined with a filler. An example of a low elasticity material 56 useful for making wall portions 55 is PEBAXT™, a polyether block amide available commercially from Archema. Other low elasticity rated engineered plastics may be used. As described herein, nanocomposites of such low elasticity materials 56 can be advantageously utilized in the wall 52 of expandable body 50. Low elasticity materials 56 can be reinforced materials such nanocomposites, filler filled materials, and irradiation crosslinked resins.

A high elasticity material 54 useful for making the wall 52 of expandable body 50 is the polyurethane TEXIN®, commercially available from Bayer MaterialScience in South Deerfield, Mass. Other materials such as silicone, rubber, thermoplastic rubbers, elastomers, and other medical balloon materials can be utilized to make high elasticity wall portions 53. Embodiments of the directionally controlled expandable body 50 can comprise a single lumen or a multi-lumen tubing of such high elasticity materials 54.

In directionally-controlled expandable bodies 50 of the present invention, distribution of pressure upon expansion is often uneven about the tubular circumference. This causes the expandable body 50 to tend to shift in a treatment area, for example, in a vertebral body 61, into regions of lower tissue density. Undesirable shifting and/or radial twisting of the expandable body 50 may also occur due to the higher elasticity of the wall 52 material. As a result, directional control of expansion can be compromised. Expandable bodies 50 having wall portions 55 of low elasticity material 56 provide greater rigidity to better maintain the expandable bodies 50 in the desired position in a treatment area. As such, expansion of bodies 50 having wall portions 55 of low elasticity material 56 can be more reliably maintained in desired locations and expanded in desired directions. As discussed herein, another advantage of wall portions 55 comprising low elasticity material 56 in a directionally-controlled expandable body 50 is greater torque control.

Moreover, the exposure of the expandable body 50 to cancellous bone 63 also typically requires materials having significant resistance to surface abrasion, puncture, and/or tensile stresses. For example, expandable bodies 50 incorporating elastomer materials, for example, polyurethane, which have been preformed to a desired shape, for example, by exposure to heat and pressure, can undergo controlled expansion and further distention in cancellous bone 63, without failure, while exhibiting resistance to surface abrasion and puncture when contacting cancellous bone 63.

Due to various pathologic or traumatic conditions, such as osteoporosis, a vertebral body 61 can compact cancellous bone 63 vertically downward and cause a decrease in height of the vertebra. A vertebral compression fracture (VCF) is a fracture occurring in a vertebra 60 which, in addition to being painful, changes the alignment of the spine. In such conditions, vertebral height is lost particularly in the anterior region of the vertebral body 60. Such a decreased height is less than the height 80 shown in FIGS. 20 and 22.

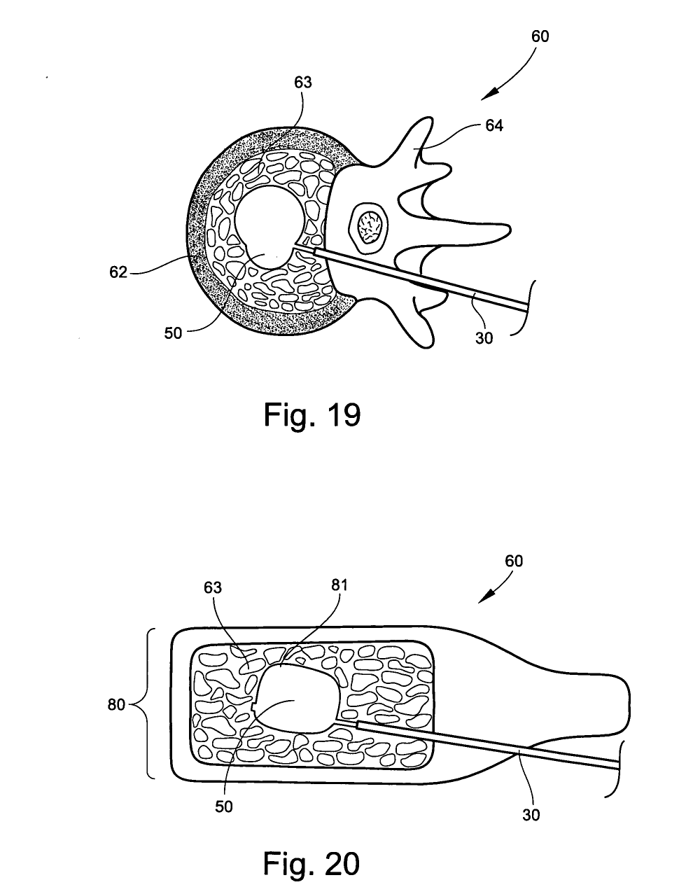

The user of the system 10, shown in FIG. 1, may wish to use the system 10 to provide a cavity 81 within the vertebral body 61, and to restore the height 80 to the vertebral body 61 lost when the fracture occurred. As shown in FIGS. 19-22, the expandable body 50 disposed at the distal end 42 of the elongate member 40 has been expanded as a result of inflation. The wall portion 53 comprising a relatively higher elasticity material 54 and the wall portion 55 comprising a relatively lower elasticity material 56 cause expansion of the expandable body 50 to be constrained more in the lower elasticity wall portion 55, resulting in expansion in the direction of the higher elasticity wall portion 53. By directing expansion of the expandable body 50 in this manner, a user of the system 10 may provide a cavity 81 having the desired dimensions. In this manner, a more normal height 80 and a pre-vertical compression fracture shape can be at least partially restored.

As shown in FIGS. 19 and 20, the expandable body 50 having the cross-section shown in FIG. 6A has been inserted through cannula 30 across pedicle 64 into cancellous bone 63 of the vertebra 60. When expanded, the expandable body 50 having this cross-section expands to the desired shape and in the desired direction as shown. The direction of expansion can be changed by the user of the system 10 by rotating the elongate member 40, and thereby the expandable body 50 disposed thereon. Using the expandable body 50 having the cross-section shown in FIG. 6A, expansion of the body 50, and compression of cancellous bone 63, can be directed vertically more in one direction than in the opposite direction as shown in FIG. 20, to increase the height of the vertebral body 61 to pre-VCF height 80.

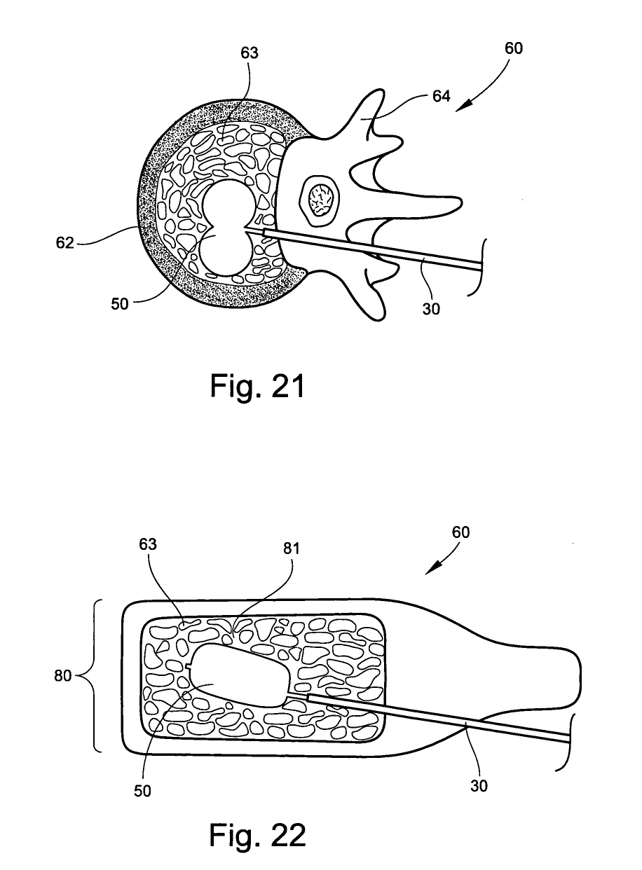

As shown in FIGS. 21 and 22, the expandable body 50 having the cross-section shown in FIG. 7A has been inserted through cannula 30 across pedicle 64 into cancellous bone 63 of the vertebra 60. When expanded, the expandable body 50 having this cross-section expands to the desired shape and in the desired direction as shown. The direction of expansion can be changed by the user of the system 10 by rotating the elongate member 40, and thereby the expandable body 50 disposed thereon. Using the expandable body 50 having the cross-section shown in FIG. 6A, expansion of the body 50, and compression of cancellous bone 63, can be directed vertically equally in both directions as shown in FIGS. 21 and 22, to increase the height of the vertebral body 61 to pre-VCF height 80.

In various embodiments, the configuration of such an expandable body 50 can be defined by the surrounding cortical bone 62 and adjacent internal structures, and is designed to occupy up to 70-90% of the volume of the inside of the bone. However, expandable bodies 50 that are as small as about 40% (or less) and as large as about 99% are workable for fractures. In various other embodiments, the expanded body 50 size may be as small as 10% of the cancellous bone 63 volume of the area of bone being treated, such as for the treatment of avascular necrosis and/or cancer, due to the localized nature of the fracture, collapse, and/or treatment area. The fully expanded size and shape of the expandable body 50 is desirably regulated by low and high durometer materials, 54, 56, respectively, in selected portions of the body 50, as described.

In embodiments of the present invention, an expandable body 50 may comprise a nanocomposite plastic material. Nanocomposites include a resin matrix and a nano-sized reinforcing filler material. Commercially available nano-fillers include clays, silicas, and ceramics. Nanocomposites and nano-fillers are available commercially from the Foster Corporation, Putnam, Conn. These fillers are small enough to improve the strength of the resin matrix, while allowing a tube to be extruded in a thin walled film.

In one embodiment, a first wall portion 53 of an expandable body 50 comprises a high elasticity material 54. A second wall portion 55 comprises a lower elasticity nanocomposite of the same material as the high elasticity wall portion 53. An advantage of using a nanocomposite material in a low elasticity wall portion 55 that is a nanocomposite of the same material used in a high elasticity wall portion 53 is that the nanocomposite material exhibits increased strength and stiffness relative to the non-reinforced material. Thus, the wall portion 55 comprising a low elasticity nanocomposite material is more resistant to stretching upon expansion of the expandable body 50 than the high elasticity wall portion 53. As a result, expansion of the expandable body 50 can be directed in desired directions according to the present invention. In an embodiment, a low elasticity, less compliant wall portion 55, or “stripe,” comprising a nanocomposite that is coextruded with a higher elasticity, more compliant wall portion 53 allows directed expansion of the expandable body 50, as described herein. In an alternative embodiment, the lower elasticity nanocomposite can be a material different than the high elasticity material 54.

Pre-determined amounts of nano-fillers in the nanocomposite can be used to selectively affect the elasticity, the degree of hardness, and the resistance to puncture, of the portions of the expandable body wall 52 comprising a nanocomposite. An advantage of using a nanocomposite material in an expandable body 50 is that relatively high elasticity resins can be used in one wall portion 53 and the same material reinforced with a nanocomposite can be used for a relatively lower elasticity wall portion 55.

In one embodiment, the entire circumference of the expandable body wall 52 is made from a nanocomposite resin. For example, a mono-layer of 100% nanocomposite resin can be extruded to make an expandable body wall 52. An expandable body 50 comprising a 100% nanocomposite resin has greater strength than an expandable body 50 made from the same resin that is not reinforced with the nanocomposite. The addition of nanocomposites to an expandable body 50 can affect the ability of the body 50 to elongate. Thus, the amount of nanocomposite used to lower the elasticity of an expandable body wall 52 should allow for sufficient elongation for achieving a desired expanded volume.

In another embodiment, an expandable body 50 is extruded as a bi-layer, comprising one layer of nanocomposite resin and the other layer of non-reinforced resin. When the outer layer of the coextruded bi-layer body 50, such as a balloon tubing 51, comprises a nanocomposite-reinforced material, the body 50 or tubing 51 is provided with increased puncture resistance. The advantage of a bi-layer extrusion is that it avoids having to use nanocomposites in 100% of the balloon tubing 51. When the entire body 50 or tubing 51 includes nanocomposites, elasticity characteristics can be affected. One way to maintain desired elasticity characteristics of a body 50 or tube 51 is to make an inner layer from a virgin material without nanocomposites and provide an outer layer, or coating, of the body 50 or tube 51 with a material comprising nanocomposites. In this way, the nanocomposite outer layer provides increased puncture resistance, while the inner layer maintains desired elasticity characteristics.

Using a nanocomposite material in the lower elasticity wall portion 55 that is a nanocomposite of the same material used in the higher elasticity wall portion 53 can improve the bond at the interface between the two wall portions 55, 53, as compared to a bond between two different materials. This provides the advantage of significantly decreasing the risk of delamination at the interface between the wall portions 55, 53. A nanocomposite provides the advantage of different material characteristics in different wall portions without compromising the interface bond between the two materials.

Utilization of a nanocomposite in an expandable body wall 52 can provide a more puncture-resistance body. Increased puncture-resistance of an expandable body 50 provides an advantage in anatomical treatment areas in which bone or other structures form sharp edges. The degree of hardness and the resistance to puncture of an expandable body wall 52 is affected by the amount of nano-fillers comprising materials different than the virgin material used in a nanocomposite. For example, if 10% of the nanocomposite comprises a nano-filler, 10% of the original molecule is replaced, causing the expandable body 50 to have 10% less of the characteristics imparted by the nanocomposite material. Inclusion of a larger percentage of nano-filler in the nanocomposite material will reduce the desired characteristics of the nanocomposite material by a proportionate larger percentage in the material. Thus, during manufacture, hardness and elasticity characteristics of a nanocomposite material in the expandable body 50 should be balanced with a desired amount of puncture-resistance.

Another advantage of the expandable body 50 of the present invention comprising a nanocomposite resin is that the very small particles of the nanocomposite allow smoother surfaces of the finished body wall 52, such as in a balloon tubing 51. In contrast, fiber-reinforced resins, which are larger, can cause imperfections in the balloon tubing 51 surface. Another advantage of the expandable body 50 of the present invention comprising a nanocomposite resin is that the body wall 52 can be thinner while achieving the same, or greater, hardness and similar elongation capabilities as in expandable bodies 50 having thicker walls 52.

As shown in the embodiment in FIG. 2, the expandable body 50 may comprise one or more radiographic markers 59 to allow radiographic visualization of the expandable body 50 in an interior body region. In alternative embodiments, the first and/or second wall portions 53, 55, respectively, of the expandable body 50 may be formed from a radiopaque material. Radiopaque is defined as being opaque to radiation and especially x-rays.

In an embodiment employing a plurality of radiographic markers 59, as shown in FIG. 2, a first set of markers 59 may be placed along the low elasticity wall portion(s) 55, where the markers 59 remain in a relatively stable position during expansion. Another set of markers 59 may be placed about the high elasticity wall portions 53 such that when the expandable body 50 is expanded, movement and positioning of the markers 59 can be visualized as the high elasticity walls 54 expand. In this manner, the size and shape of the expanded body 50, and the cavity 81 (FIGS. 20, 22, and 24), can be visualized.

Radiopaque materials useful for inclusion in the walls of the expandable body 50 include, for example, barium sulfate, tantalum, tungsten, and bismuth subcarbonate. A powder of such radiopaque materials can be compounded with selected low elasticity and/or high elasticity materials 56, 54 for making expandable bodies 50 and extruded together with the selected materials to form a tube. Alternatively, radiopaque materials can be extruded as wires and arranged in different lumens of the cannula 30 such that the expandable body 50 can be visualized under a fluoroscope.

In other embodiments, other means for radiographic visualization of the expandable body 50 can be used. For example, the location, size, and shape of the expandable body 50 can be visualized under fluoroscopy by expanding the body 50 with a radiopaque gas or liquid.

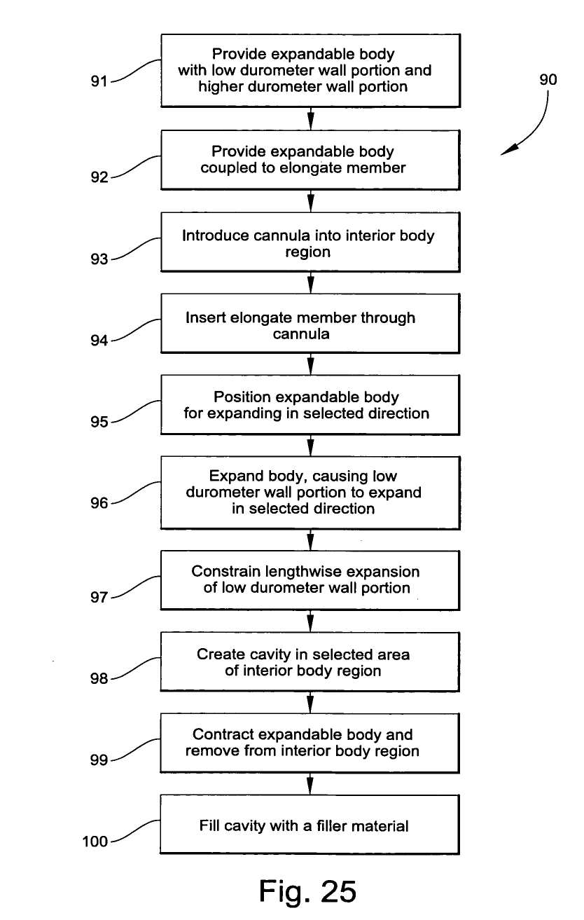

Embodiments of the present invention include methods for directionally controlling expansion of an expandable body 50 in a targeted treatment area. One such method 90 is shown in the flow chart in FIG. 25. With reference also to FIGS. 1-2, the expandable body 50 is provided (91) with a wall 52 having a first wall portion 53 comprising a high elasticity material 54 and a second wall portion 55 comprising a material 56 having an elasticity lower than the elasticity of the first wall portion 53. The expandable body 50 is coupled (92) to the distal end 42 of the elongate member 40. The cannula 30 is introduced (93) into an interior body region. The elongate member 40 is then inserted (94) through the cannula 30. Once the expandable body 50 can be positioned (95) for expanding in a selected direction in the interior body region, the expandable body is expanded (96) by injecting a flowable material. The expandable body 50 comprises an elongated axis 58, and causing directed expansion (96) of the body 50 causes the first wall portion 53 to expand outwardly 57 in the selected direction along the elongated axis 58.

In such an embodiment of the method 90, causing directed expansion (96) of the body 50 causes the first wall portion 53 to expand in a constrained manner (97) lengthwise along the elongated axis 58. In embodiments, the directed expansion (96) creates (98) a cavity 81 within the interior body region. The interior body region may comprise a bone, including, for example, a cancellous bone 63, which is compressed by the directed expansion (96). In an embodiment, the directed expansion (96) displaces a cortical bone 62. The directed expansion (96) may be utilized to intervene in other interior body regions. For example, the directed expansion (96) may be utilized to lift vertebral end plates, tibial plateau depressions, and proximal humerus depressions, as well as for other purposes.

In an embodiment, the method 90 includes contracting (99) the expandable body 50 and 4 removing the expandable body 50 from the interior body region. In another embodiment, the method 90 can include filling (100) the cavity 81 with a filler material.

The various embodiments of expandable bodies 50 disclosed herein are by no means limited in their utility to use in a single treatment location within the body. Rather, while each embodiment may be disclosed in connection with an exemplary treatment location, these embodiments can be utilized in various locations within the human body, depending upon the treatment goals as well as the anatomy of the targeted bone. For example, embodiments of an expandable body 50 may be used in the treatment of areas within the body other than the vertebra, including, for example, the ribs, the femur, the radius, the ulna, the tibia, the humerus, the calcaneus, or the spine. As an example, particular embodiments of such expandable bodies 50 may be utilized to lift, for example, tibial plateau depressions and proximal humeral depressions.

Although the present invention has been described with reference to particular embodiments, it should be recognized that these embodiments are merely illustrative of the principles of the present invention. Those of ordinary skill in the art will appreciate that a directionally controlled expandable device and methods of use of the present invention may be constructed and implemented in other ways and embodiments. Accordingly, the description herein should not be read as limiting the present invention, as other embodiments also fall within the scope of the present invention.

What is claimed is:

1. A device comprising:
an expandable body comprising a wall having a first wall portion comprising a high elasticity material and a second wall portion comprising a material having an elasticity lower than the first wall portion elasticity.

2. The device of claim 1,
wherein expansion of the second wall portion is constrained more than expansion of the first wall portion, and
wherein expansion of the body is directed outwardly from the first wall portion.

3. The device of claim 1,
wherein the expandable body comprises an elongated axis, and
wherein the second wall portion constrains expansion of the body lengthwise along the elongated axis.

4. The device of claim 1,
wherein the expandable body comprises an elongated axis, and
wherein the first wall portion and the second wall portion extend along the elongated axis.

5. The device of claim 1, wherein the expandable body wall comprises a plurality of the first wall portions and a plurality of the second wall portions.

6. The device of claim 1, wherein the high elasticity material comprises a low durometer material and the lower elasticity material comprises a high durometer material.

7. The device of claim 1, the expandable body further comprising an internal restraint coupled to the body for directing expansion of the body in opposite directions.

8. The device of claim 1, further comprising a substantially rigid surface adjacent the expandable body,
wherein the substantially rigid surface resists displacement during expansion of the body, and
wherein the body is expanded in one direction away from the substantially rigid surface.

9. The device of claim 1, wherein a thickness of the second wall portion is greater than a thickness of the first wall portion.

10. The device of claim 1, wherein the expandable body wall comprises a third wall portion comprising a material having an elasticity lower than the first wall portion elasticity and different than the second wall portion elasticity.

11. The device of claim 1, wherein the second wall portion extends through a full thickness of the body wall.

12. The device of claim 1, wherein the second wall portion extends through a partial thickness of the body wall.

13. The device of claim 1, wherein the expandable body wall comprises a non-circular cross-section.

14. The device of claim 1, wherein the expandable body comprises an inflatable balloon tube.

15. The device of claim 14, wherein the inflatable balloon tube comprises multiple lumens.

16. The device of claim 1, wherein the second wall portion comprises the lower elasticity material and a nanocomposite of the lower elasticity material.

17. The device of claim 16, wherein the nanocomposite comprises a nano-filler comprising a material other than the low elasticity material.

18. The device of claim 1, wherein the first wall portion comprises a radiopaque material.

19. The device of claim 1, wherein the first wall portion comprises a radiopaque material.

20. The device of claim 1,
wherein the expandable body wall comprises a substantially circular cross-section and an elongated axis,
wherein the body wall comprises the second wall portion on one side of the body wall cross-section, and
wherein when the body is expanded, the body curves at an angle from the elongated axis.

21. The device of claim 20, wherein the angle the body curves from the elongated axis is in the range of 30-90 degrees.

22. The device of claim 1,
wherein the expandable body wall comprises a substantially circular cross-section and an elongated axis,
wherein a thickness of the body wall on one side of the body wall cross-section is greater than a thickness of the body wall on an opposite side of the body wall cross-section, and
wherein when the body is expanded, the body curves at an angle from the elongated axis.

23. The device of claim 1,
wherein the expandable body wall comprises a substantially circular cross-section and an elongated axis,
wherein one side of the body wall cross-section comprises a non-compliant material, and
wherein when the body is expanded, the body curves at an angle from the elongated axis.

24. A system comprising:
a cannula comprising a cannula distal end;
an elongate member comprising an elongate member distal end; and
an expandable body coupled to the elongate member distal end and configured to be slidably disposed within the cannula, the expandable body comprising a wall having a first wall portion comprising a high elasticity material and a second wall portion comprising a material having an elasticity lower than the first wall portion elasticity.

25. The system of claim 24,
wherein expansion of the second wall portion is constrained more than expansion of the first wall portion, and
wherein expansion of the body is directed outwardly from the first wall portion.

26. The system of claim 24,
wherein the expandable body comprises an elongated axis, and
wherein the second wall portion constrains expansion of the body lengthwise along the elongated axis.

27. The system of claim 24,
wherein the expandable body comprises an elongated axis, and
wherein the first wall portion and the second wall portion extend along the elongated axis.

28. The system of claim 24, wherein the high elasticity material comprises a low durometer material and the lower elasticity material comprises a high durometer material.

29. The system of claim 24, wherein the second wall portion comprises the lower elasticity material and a nanocomposite of the lower elasticity material.

30. The system of claim 24, wherein the first wall portion comprises the high elasticity material and a first amount of a nanocomposite of the high elasticity material and
the second wall portion comprises the high elasticity material and a second amount of the nanocomposite of the high elasticity material,
wherein the second amount of the nanocomposite is larger than the first amount of the nanocomposite.

31. The system of claim 24, wherein the first wall portion comprises a radiopaque material.

32. A method comprising:
providing an expandable body comprising a wall having a first wall portion comprising a high elasticity material and a second wall portion comprising a material having an elasticity lower than the first wall portion elasticity.

33. The method of claim 32, wherein providing the expandable body comprises providing the high elasticity material comprising a low durometer material and the lower elasticity material comprising a high durometer material.

34. The method of claim 32, wherein the providing the expandable body further comprises coextruding the first wall portion and the second wall portion.

35. The method of claim 32,
wherein the expandable body comprises an elongated axis, and
wherein the first wall portion and the second wall portion are coextruded to extend along the elongated axis.

36. The method of claim 32, wherein the second wall portion comprises the lower elasticity material and a nanocomposite of the lower elasticity material.

37. The method of claim 32, wherein providing the expandable body comprises providing
the first wall portion comprising the high elasticity material and a first amount of a nanocomposite of the high elasticity material and
the second wall portion comprising the high elasticity material and a second amount of the nanocomposite of the high elasticity material,
wherein the second amount of the nanocomposite is larger than the first amount of the nanocomposite.

38. The method of claim 32, wherein the first wall portion comprises a radiopaque material.

39. A method comprising:
providing an expandable body coupled to a distal end of an elongate member, the expandable body comprising a wall having a first wall portion comprising a high elasticity material and a second wall portion comprising a material having an elasticity lower than the first wall portion elasticity;
introducing a cannula into an interior body region;
inserting the elongate member through the cannula such that the expandable body is positioned for expanding in a selected direction in the interior body region; and
causing directed expansion of the expandable body,
wherein the first wall portion expands in the selected direction.

40. The method of claim 39,
wherein the expandable body comprises an elongated axis, and
wherein causing directed expansion of the body causes the first wall portion to expand outwardly in the selected direction along the elongated axis.

41. The method of claim 39,
wherein the expandable body comprises an elongated axis, and
wherein causing directed expansion of the body causes the first wall portion to expand in a constrained manner lengthwise along the elongated axis.

42. The method of claim 39, wherein the directed expansion creates a cavity within the interior body region.

43. The method of claim 39, wherein the interior body region comprises a bone.

44. The method of claim 43, wherein the causing directed expansion of the body comprises compressing a cancellous bone within the bone.

45. The method of claim 43, wherein the directed expansion displaces a cortical bone.

46. The method of claim 43, wherein the directed expansion lifts vertebral end plates.

47. The method of claim 43, wherein the directed expansion lifts tibial plateau depressions.

48. The method of claim 43, wherein the directed expansion lifts proximal humerus depressions.
